(19) United States
(12) Patent Application Publication
(10) Pub. No.: US 2007/0036866 A1
(43) Pub. Date: Feb. 15, 2007

(54) Cationic block copolymers

(76) Inventors: Thomas Kissel, Staufen (DE); Holger Petersen, Marburg (DE); Dagmar Fischer, Marburg (DE); Klaus Kunath, Marburg (DE); Anke Von Harpe, Marburg (DE)

Correspondence Address: PROPAT, L.L.C., 425-C SOUTH SHARON AMITY ROAD, CHARLOTTE, NC, 28211-2841, US

(21) Appl. No.: 11/582,679

(22) Filed: Oct. 18, 2006

Related U.S. Application Data
(62) Division of application No. 10/030,803, filed on Apr. 9, 2002.

(30) Foreign Application Priority Data
Jul. 15, 1999 (DE) 199 33 024.7

(51) Int. Cl. A61K 48/00; C08L 71/12; A61K 9/14

(52) U.S. Cl. 424/486; 525/396; 514/44

(57) ABSTRACT

The invention relates to cationic block copolymers of formula A(—X—B)ₙ or C(—Y—D)ₘ, wherein A represents a hydrophilic polymer, B represents polyethyleneimine (PEI), X represents a bridge, n represents 1-200, C represents PEI, D represents the residue of a polyethylene glycol, Y represents a bridge and m represents 1-200. The invention also relates to methods for producing the inventive cationic block polymers and to their use e.g. as a tenside and for complexing nucleic acids.

### CROSS-REFERENCE TO RELATED APPLICATIONS

This divisional application claims priority to parent application Ser. No. 10/030,803, filed Apr. 9, 2002, hereby incorporated by reference herein in its entirety. This application further claims priority to PCT/EP00/06214, filed Jul. 4, 2000, which claims priority to German Patent Application No. 199 33 024.7, filed Jul. 15, 1999. Both International Application No. PCT/EP00/06214 and German Patent Application No. 199 33 024.7 are hereby incorporated by reference herein in their entirety.

### BACKGROUND OF THE INVENTION

WO 98/59064 discloses PEI-PEG block copolymers and their use as vehicles for transporting nucleic acid into higher eukaryotic cells. The described copolymer was composed of branched PEI and linear PEG. PEI was PEGylated using methoxy-succinimidyl-propionate-PEG.

S. V. Vinogradov, T. K. Bronich and A. V. Kabanov (Bioconjugate Chem. 1998, 9, 805-812) describe the preparation of PEI-PEG and polyspermine-PEG block copolymers by using branched PEI and branched polyspermines through a coupling reaction with a monomethoxy-PEG activated with 1,1′-carbonyldiimidazole. The copolymers were used for complexation with oligonucleotides.

L. M. Bronstein, M. Antonietti et al. (Inorganica Chimica Acta 1998, 280, 348-354) describe PEI-PEG block copolymers and their preparation by coupling of branched PEI with monomethoxy-PEG which has a terminal acid chloride function, and the use thereof for preparing metal colloids.

V. Toncheva et al. (Biochimica et Biophysika Acta 1998, 138, 354-358) relates to block copolymers consisting of poly(L-lysine) and a plurality of hydrophilic polymers, such as PEG, dextran and poly(N-(2-hydroxypropyl)-methacrylamide, processes for their preparation and their use as vehicles for nucleic acid gene transfer.

These known block copolymers have the following three points in common:
1. The cationic polymer is equipped with side arms of a hydrophilic, nonionic polymer.
2. In all cases, for this purpose the reactive terminus of the hydrophilic, nonionic polymer was activated for the coupling reaction with the cationic polymer by a reagent which represents a linker between the blocks in the copolymer which is generated.
3. The hydrophilic, nonionic polymer was in all cases a linear polymer.

### SUMMARY OF THE INVENTION

Novel cationic block copolymers of the general formula I and II
A(—X—B)n (I)
C(—Y—D)ₘ (II)
have been found, in which
A is a hydrophilic, nonionic, linear or branched polymer with a molecular weight of from 100 to 10 000 000 g/mol, preferably from 1000 to 100 000 g/mol and in particular from 5000 to 50 000 g/mol;
B is a linear or branched polyethyleneimine (PEI) with a molecular weight of from 100 to 1 000 000 g/mol, preferably from 400 to 100 000 g/mol and in particular from 400 to 50 000 g/mol;
X is a direct linkage of blocks A and B or a linker with the following structures:

—OC(O)NH(CH₂)ₒNHC(O)NH— with o=1 to 20, preferably 2 to 10, in particular 4 to 6,

—OC(O)NH(aryl)NHC(O)NH— with aryl=aromatic unit with, preferably, 6-14 C atoms consisting of one or more aromatic nuclei which are connected together in fused or in polyphenylic form, preferably with one nucleus, in particular tolyl,

—O(CH₂)PC(O)NH— with p=1 to 10, preferably 1 to 3, in particular 1,

—OCH₂CH(OH)CH₂NH—,

—OC(O)NH—, or

—O(CH₂)_{q}NH— with q=1 to 20, preferably 1 to 6, in particular 1 to 3;

n is an integer from
1) 1 to 200, preferably
2) 1to 50,
3) 1 to 12,
4) 1 to 8 or, particularly preferably,
5) 2 to 8;
C is a linear or branched PEI with a molecular weight of from 100 to 1 000 000 g/mol, preferably from 400 to 100 000 g/mol, and in particular from 400 to 50 000 g/mol;
D is a residue of a polyethylene glycol which is linked via O of the formula
   —(CH₂CH₂O)ₙ—R¹
   in which n′ is from 3 to 25 000, preferably from 10 to 5000 and in particular from 10 to 1000, and R¹ is hydrogen, an aliphatic radical such as (C₁-C₆)-alkyl (methyl, ethyl, tert-butyl and the like) or another OH-protective group such as acyl (e.g. optionally substituted benzoxy-carbonyl), optionally substituted benzyl, picolyl, or a cellular ligand in order to bring about specific uptake of a nucleic acid-copolymer complex through binding to cell surface proteins, in particular receptors;
Y is a direct linkage of blocks C and D or a linker with the following structures:

—NHC(O)NH(CH₂)ₛNHC(O)O— with s=1 to 20, preferably 2 to 10, in particular 4 to 6,

—NHC(O)NH(aryl)NHC(O)O— with aryl=aromatic unit with, preferably, 6 to 14 C atoms consisting of one or more aromatic nuclei which are connected together in fused or in polyphenylic form, preferably with one nucleus, in particular tolyl,

—NH(CH₂)ₜC(O)O— with t=2to 10, preferably 2 to 3, in particular 2,

—NHCH₂CH(OH)CH₂O—, or

NH(CH₂)ᵤO— with u=1 to 20, preferably 1 to 6, in particular 1 to 3; and

m is an integer from
1) 1 to 200, preferably
2) 1 to 100, in particular
3) 1 to 50.

### DETAILED DESCRIPTION OF THE INVENTION

The cationic block copolymers of the invention differ from the known block copolymers in at least one of the following three features:
1. A hydrophilic, nonionic polymer is equipped with side arms of a cationic polymer.
2. The linkers differ from those of known block copolymers.
3. They have a branched hydrophilic, nonionic polymer.

A preferably means linear or branched polymers which are composed of carbon and oxygen and which may, where appropriate, also comprise cyclic, star or dendritic structures, such as, for example, residues of linear PEG, multi-arm branched PEG, star PEG, polysaccharides including cyclodextrins, PVA, arborols (dendrimers with terminal hydroxyl groups), but preferably linear and multi-arm branched and star PEGs. The latter are commercially available inter alia from Aldrich, Fluka, Sigma and Shearwater.

B and C mean linear or branched polyethyleneimine residues which have the formula III
—[CH₂CH₂N^{(z+)}(R²)ₓ]_{y}—H[A⁻]_{w} (III)
in which R² is identical or different radicals and is hydrogen or a radical of the formula IV
—[CH₂CH₂N^{(z′+)}(R³)_{x′}]_{y′}—H[A⁻]_{w′} (IV)

R³ is identical or different radicals which (recursively) are defined as R²,

A⁻ are an equivalent of a suitable, preferably inorganic anion such as OH⁻, Cl⁻, Br⁻ and the like,

x and x′ are identical or different and are 1 or 2,

y and y′ are identical or different and are integers which are chosen so that the radicals B and C have a constituent molecular weight of from 100 to 1 000 000 g/mol, preferably from 400 to 100 000 g/mol and in particular from 400 to 50 000 g/mol, it also being possible for y′ to be 0,

z and z′ are identical or different, z=x−1 and z′=x′−1 and

w and w′ are identical or different integers which are chosen so as to balance the positive charges in the radicals of the formulae III and IV.

The polyethyleneimines can be prepared in a manner known per se or are commercially available under the BASF brand name Lupasol® or under the name polyethyleneimine or ethyleneimine polymer in various molecular weights of from 400 to 2 000 000 g/mol (from Aldrich, Sigma, Fluka or directly from BASF). Preference is given to polyethyleneimines with a molecular weight of from 400 to 2000 g/mol for B and to polyethyleneimines with a molecular weight of from 400 to 800 000 g/mol, particularly preferably from 400 to 25 000 g/mol, for C.

The groups described under D are residues of polyethylene glycols which are protected on one terminus by a radical R¹ such as, for example, methyl or another suitable protective group. However, R¹ may also be a group which performs a specific or nonspecific biological function, in particular a ligand for interactions with receptors for target cell-specific uptake of a block copolymer-active substance complex into higher eukaryotic cells and the cell nucleus thereof, where the active substance is preferably an oligonucleotide or a gene (gene targeting). R¹ can thus also be a ligand for a specific interaction and uptake into target organ tissue or cells, for example proteins, in particular antibodies or antibody fragments such as Fab, F(ab)₂, scFv, cytokines or lymphokines, such as interleukins (IL-2 to x), interferon GM-CSF, growth factors such as EGF, PDGF, FGF, EPO, integrins such as ICAM, VCAM or glycoproteins such as lectins or glycosilated proteins (see above) or lipoproteins such as LDL, HDL or transporter proteins such as transferrin or peptides such as LH-RH, calcitonin, oxytocin, insulin, somatostatin, IGF, RGD or carbohydrates such as galactose, mannose, glucose, lactose or hormones such as steroids, THR or vitamins such as B₁₂, folic acid.

The invention also relates to processes for preparing compounds of the formula I, which comprise

a) reacting compounds of the general formula V
A—(OH)ₙ with A and n=as in formula I (V)
with diisocyanate, preferably hexamethylene diisocyanate, and reacting the compound resulting therefrom with polyethyleneimines with the general formulae III and IV, or

b) adding compounds of the general formula VI
A—(NH₂)ₙ (with A and n=as defined in formula I) (VI)
to the reaction mixture for the polymerization of ethyleneimine before the start of the polymerization or not until the polymerization is in progress, or

c) employing compounds of the general formula VII
A—(OS(O)₂R⁴)ₙ with A as in formula I and R⁴=aliphatic or aromatic radical, preferably p-tolyl, fluoride, trifluoromethyl or methyl, (VII)
as macroinitiator for the polymerization of ethyleneimine.

Compounds of the formula VI are commercially available in various molecular weights, for example from Shearwater.

Compounds of the general formula VII are obtained by reacting compounds of the general formula V with compounds of the general formula VIII
Cl—S(O)₂R⁴ (R⁴ as defined above). (VIII)

The invention further relates to processes for preparing compounds of the formula II, which comprise

d) initially reacting compounds of the general formula IX
D—OH (with D as defined in formula II) (IX)
with diisocyanate, preferably hexamethylene diisocyanate, and subsequently reacting the resulting compound with linear or branched polyethyleneimine. Protective groups introduced where appropriate to protect OH groups can be eliminated in a manner known per se (see, for example, Büllesbach, Kontakte (Merck) 1/1980, pp. 23 et seq.).

The process described under a) is preferably carried out in such a way that a 4- to 20-fold excess of diisocyanate, preferably hexamethylene diisocyanate, is employed per terminal hydroxyl group of the polymer block A. The reaction is carried out in chloroform at temperatures from room temperature to the boiling point of the solvent, but preferably at the boiling point of the solvent. The chosen reaction time is between 2 and 24 hours, but preferably 4 hours. The polymer concentration in the reaction mixture is between 10 g/l and 500 g/l, preferably 100 g/l. The product is isolated by removing the solvent under reduced pressure and removing the excess diisocyanate by repeated extraction with petroleum ether (boiling range: 40-60° C.). This intermediate is reacted with a 3- to 10-fold excess of PEI macromolecules per terminal hydroxyl group of the starting compound. The reaction is carried out in chloroform at temperatures from room temperature to the boiling point of the solvent, but preferably at the boiling point of the solvent. The chosen reaction time is between 6 and 72 hours, but preferably 12 hours. The polymer concentrations, both those of the PEI and those of the nonionic hydrophilic polymer which has been activated with hexamethylene diisocyanate, in the reaction mixture are between 10 g/l and 500 g/l, preferably between 30-200 g/l. The product is isolated by precipitating the polymer in a 10-30-fold volumetric excess of diethyl ether. Excess PEI can be removed from the block copolymer by repeated reprecipitation with ethanol and diethyl ether as solvent.

The process described under b) is carried out by mixing the ethyleneimine and the amino-terminated hydrophilic nonionic polymer in water in a concentration of from 10 g/l to 500 g/l in each case. The molar ratio of the two components is between 1:10 to 1:10 000. The ethyleneimine polymerization is then initiated by adding a suitable catalyst, for example hydrochloric acid, and the mixture is brought to a temperature of 40-100° C. The copolymer is generated by a chain termination reaction. The block copolymer is repeatedly reprecipitated with the aid of suitable solvents, for example ethanol and diethyl ether, and/or by pressure filtration, to remove PEI homopolymer which is a possible by-product. One variation of this preparation process comprises adding the amino-terminated hydrophilic nonionic polymer to the hot polymerization mixture only after a certain reaction time of from 30 minutes up to 72 hours.

The process described under c) is carried out by reacting the terminal hydroxyl group(s) of the polymer block A with a sulfonyl chloride of the general formula VIII, but especially with toluenesulfonyl chloride (tosyl chloride). This reaction is carried out in aqueous and/or polar organic solvent, preferably in a water/tetrahydrofuran mixture, at temperatures of from −10° C. to the boiling point of the solvent, preferably at temperatures of from −10° C. to the boiling point of the solvent, preferably at temperatures of from 0° C. to 25° C., and (if necessary) in the presence of catalysts such as, for example, triethylamine or sodium hydroxide. The product is isolated by removing the solvent under reduced pressure. This polymer is subsequently used as macroinitiator for the ethyleneimine polymerization. For this purpose, the product with the general formula VII is reacted with ethyleneimine in aqueous or polar organic solvent at temperatures of from 0° C. to the boiling point of the solvent. The molar ratio of the two components is between 1:10 to 1:10 000. No by-product is formed in this reaction. The final product can be isolated by precipitating the polymer in a suitable solvent such as, for example, diethyl ether. The process described under d) is preferably carried out by reacting a compound of the general formula IX with a small excess, preferably a 2- to 10-fold excess, of diisocyanate, preferably hexamethylene diisocyanate. The reaction is carried out in chloroform at temperatures of from 20° C. to the boiling point of the solvent, but preferably at the boiling point of the solvent. The chosen reaction time is between 2 and 24 hours, but preferably 10 to 14 hours. The polymer concentration in the reaction mixture is between 10 g/l and 500 g/l, preferably 30 to 150 g/l. The product is isolated by removing the solvent under reduced pressure and removing the excess diisocyanate by repeated extraction with petroleum ether (boiling range: 40-60° C.). This intermediate is reacted with PEI macromolecules in a molar ratio of from 1:1 to 100:1. The reaction is carried out in chloroform and, if necessary, with addition of dimethylformamide at temperatures from room temperature to the boiling point of the solvent, but preferably at 60-70° C. The chosen reaction time is between 6 and 72 hours, but preferably 12 hours. The polymer concentrations, both those of the PEI and those of the nonionic hydrophilic polymer activated with hexamethylene diisocyanate, in the reaction mixture are between 10 g/l and 500 g/l, preferably between 30-200 g/l. The product is isolated by precipitating the polymer in a 10-30-fold volumetric excess of diethyl ether.

Compared with PEI, the novel compounds have the following properties:

The block copolymers have a lower toxicity than PEI homopolymers in cytotoxicity tests and remain longer in the blood circulation (see “Biological Examples” section).

The block copolymers are more or less, depending on the structure, surface-active substances which can be used as surfactants.

In addition, the block copolymers can also be used
in adhesive and coating systems as additive
as fixing agents to improve paper strength
as primers for polymer composite systems such as, for example, multilayer packaging sheets
for modifying plastics (improving the dyeability, paintability, barrier effect)
for fixing reactive dyes on cotton
as coagulant and dispersant for fine suspended particles in industrial waste waters
for binding heavy metal salts
for dispersing organic and inorganic pigments
as addition in ceramic and cement components
for a wide variety of functions in skin and hair cosmetics and in the dental sector
for immobilizing medicinal active substances or bioactive compounds on surfaces
for filtering endotoxins and pathogens out of blood plasma
for penetration through mucous membranes.

In addition, in aqueous systems the block copolymers form complexes with polynucleic acids such as DNA and RNA, including ribozymes. This property makes them suitable as vehicles or vectors for gene transfer (penetration through cell membranes and translocation into the cell nucleus). They can therefore be used in transfection experiments, in gene therapy and diagnosis (see “Biological Examples” section).

The following examples serve to illustrate the invention without intending to restrict it thereto.

### CHEMICAL EXAMPLES

### Example 1

### Preparation of a PEI(PEG)ₙ Block Copolymer

Activation of mPEG-550

10 ml of chloroform are introduced into a 100 ml round-bottomed flask with magnetic stirring bar, reflux condenser and drying tube on top, and 7 ml of hexamethylene diisocyanate (HMDI) (43.64 mmol, 8 eq.) are added. 3 g of polyethylene oxide monomethyl ether (mPEG, Mₙ=550 g/mol) (5.45 mmol, 1 eq.) are dissolved in 40 ml of chloroform. This solution is then slowly added dropwise to the stirred HMDI solution. The mixture is heated under reflux for 12 hours. The solvent is then removed under reduced pressure, and the excess HMDI is extracted with petroleum ether (40-60) (5×50 ml). The product is obtained as a colorless mobile oil in virtually quantitative yield (3.8 g, 97%).

### Preparation of a PEI-graft-PEG Block Copolymer

1.74 g of bPEI (M_{w}=25 kDa, Mₙ=10 kDa, 0.1736 mmol, 1 eq.) are weighed into a 100 ml round-bottomed flask with magnetic stirring bar, reflux condenser and drying tube on top, and dissolved in 40 ml of dimethylformamide (DMF). 2.5 g of the HMDI-activated mPEG (Mₙ=720 Da, 3.47 mmol) are dissolved in 10 ml of chloroform, and this solution is slowly added dropwise to the stirred PEI solution. The mixture is heated at 60-70° C. for 12 hours. The mixture is then added dropwise to 500 ml of diethyl ether. After two hours, a viscous yellowish oil has deposited. The cloudy supernatant is discarded, and the oil is dissolved in 30 ml of ethanol. The solution is again added dropwise to 500 ml of diethyl ether, and the oil which has again separated out is isolated by decantation. The product is dissolved in ethanol for filtration, and the solvent is removed in a vacuum oven at 50° C. 2.8 g of a yellowish viscous to resinous oil are obtained (yield: 45%).

The polymers were characterized by ¹H and ¹³C NMR spectroscopy and gel permeation chromatography. The following data were obtained for Example No.1. They are representative of the other examples, for which similar data were obtained.

¹H NMR (500 MHz, CDCl₃): δ/ppm=1.17 (isocyanate CH₂), 1.26 (isocyanate CH₂), 2.30-2.72 (ethyleneimine CH₂),2.96 (isocyanate CH₂), 3.15 (isocyanate CH₂), 3.49 (ethylene glycol CH₂). ¹³C NMR (125 MHz, CDCl₃): δ/ppm 14.3 (isocyanate CH₂), 26.2 (isocyanate CH₂), 29.6 (isocyanate CH₂), 36.2 (isocyanate CH₂), 37.5 (ethyleneimine CH₂), 39.1 (ethyleneimine CH₂), 41.1 (isocyanate CH₂), 47.2 (ethyleneimine CH₂), 48.9 (ethyleneimine CH₂), 52.8 (ethyleneimine CH₂), 54.1 (ethyleneimine CH₂), 58.7 (isocyanate CH₂), 69.3 and 70.2 and 71.6 (ethylene glycol CH₂), 156.2 (—NHC(O)O—), 161.7 (—NHC(O)NH—).

GPC (aminoethyl methacrylate gel, 1% formic acid, 0.5 ml/min, 25° C., calibrated using pullulan standards): Mₙ=8800, M_{w}=1 640 000, Mₚ=85 000, PD=19.6, monomodal.

Comparison with blend of PEI (Aldrich, 25 kDa) and mPEG (Aldrich, 550 Da): Mₙ=69 000, M_{w}=1 480 000, Mₚ=99 000 and 1100, PD=2.1, bimodal.

Investigations of the surface activity of the polymer of Example No. 1 were carried out by the method of Lecomte du Nouy (ring method) at 22° C. using a tensiometer. The surface tension of the solution in relation to air was measured. The instrument was calibrated with extra pure water, which was also employed as solvent for the polymer sample.

Measured data: σₘᵢₙ=51 mN/m, CMC=15 mg/ml.

The following can be prepared in the same way: (all starting compounds are obtainable from Aldrich)

| | Starting compounds/homopolymers | | Molar | Structure |
|---|---|---|---|---|
| | Polyethyl- | Hydrophilic, | ratio | of the block |
| No. | eneimine | nonionic polymer | PEI:PEG | copolymer |
| 2 | IPEI Mₙ | mPEG Mₙ | 1:1 | AB diblock |
| 3 | ca. 423 | ca. 550 | 1:2 | ABA triblock |
| 4 | bPEI M_{w} | mPEG Mₙ | 1:1 | AB diblock |
| 5 | ca. 800 | ca. 550 | 1:2 | ABA triblock |
| 6 | | | 1:4 | star |
| 7 | bPEI M_{w} | mPEG Mₙ | 1:1 | AB diblock |
| 8 | ca. 800 | ca. 5000 | 1:2 | ABA triblock |
| 9 | | | 1:4 | star |
| 10 | bPEI M_{w} | mPEG Mₙ | 1:1 | AB diblock |
| 11 | ca. 2000 | ca. 550 | 1:2 | ABA triblock |
| 12 | | | 1:4 | star |
| 13 | bPEI M_{w} | mPEG Mₙ | 1:1 | AB diblock |
| 14 | ca. 2000 | ca. 5000 | 1:2 | ABA triblock |
| 15 | bPEI M_{w} | mPEG Mₙ | 1:1 | AB diblock |
| 16 | ca. 25 000 | ca. 550 | 1:2 | ABA triblock |
| 17 | | | 1:4 | star |
| 18 | | | 1:20 | star |
| 19 | bPEI M_{w} | mPEG Mₙ | 1:1 | AB diblock |
| 20 | ca. 25 000 | ca. 2000 | 1:2 | ABA triblock |
| 21 | | | 1:4 | star |
| 22 | | | 1:10 | star |
| 23 | bPEI M_{w} | mPEG Mₙ | 1:1 | AB diblock |
| 24 | ca. 25 000 | ca. 5000 | 1:2 | ABA triblock |
| 25 | | | 1:4 | star |
| 26 | | | 1:10 | star |

### Example 27

### Preparation of a PEG(PEI)ₙ Block Copolymer

Activation of Branched PEG

3.79 g of HMDI (22.54 mmol, 80 eq.) are dissolved in 10 ml of chloroform in a 100 ml round-bottomed flask with magnetic stirring bar, reflux condenser and drying tube on top. A solution of 2 g of an eight-arm branched PEG (bPEG, MW=10 kDa, 0.2 mmol, 1 eq.) in 20 ml of chloroform is slowly added dropwise to the stirred HMDI solution. The mixture is boiled for 4 hours and then stirred at room temperature for a further 8 hours. The solvent is removed under reduced pressure, and the excess HMDI is extracted with petroleum ether (40-60) (3×50 ml). A reddish oil is obtained in a yield of 58% (1.38 g).

### Preparation of a PEG-graft-PEI Block Copolymer

2.20 g of a branched PEI (bPEI, M_{w}=800 Da, Mₙ=600 Da, 3.66 mmol, 25 eq.) are dissolved in 20 ml of chloroform in a 100 ml round-bottomed flask with magnetic stirring bar, reflux condenser and drying tube on top. A solution of 1.21 g of the HMDI-activated bPEG (Mₙ=8.5 kDa, 0.14 mmol, 1 eq.) in 30 ml of chloroform is slowly added dropwise to the stirred PEI solution at room temperature. The mixture is boiled for 12 hours. The solution is then slowly added dropwise to 500 ml of diethyl ether while stirring. After 12 hours, a viscous yellowish oil has deposited. The cloudy supernatant is discarded, and the oil is dissolved in 50 ml of ethanol. The solution is again added dropwise to 500 ml of diethyl ether, and the oil which has again separated out is isolated by decantation. The product is dissolved in ethanol for filtration, and the solvent is removed in a vacuum oven at 50° C. 1.13 g of a yellowish viscous to resinous oil are obtained (yield: 59%).

The polymers were characterized by ¹H and ¹³C NMR spectroscopy and gel permeation chromatography. The following data were obtained for Example No.27. They are representative of the other examples, for which similar data were obtained.

¹H NMR (500 MHz, CDCl₃): δ/ppm=1.22 (isocyanate CH₂), 1.36 (isocyanate CH₂), 2.40-2.70 (ethyleneimine CH₂), 3.03 (isocyanate CH₂),3.19 (isocyanate CH₂), 3.55 (ethylene glycol CH₂).

¹³C NMR (125 MHz, CDCl₃): δ/ppm=25.9 (isocyanate CH₂), 29.4 (isocyanate CH₂), 39.2 (ethyleneimine CH₂), 41.2 (isocyanate CH₂), 47.0 (ethyleneimine CH₂), 48.9 (ethyleneimine CH₂), 52.0 (ethyleneimine CH₂), 54.2 (ethyleneimine CH₂), 61.1 (isocyanate CH₂), 69.2 and 71.1 and 72.3 (ethylene glycol CH₂), 156.0 (—NHC(O)O—), 162.1 (—NHC(O)NH—).

GPC (aminoethyl methacrylate gel, 1% formic acid, 0.5 ml/min, 25° C., calibrated using pullulan standards): Mₙ=22 000, M_{w}=43 000, Mₚ=31 000, PD=1.9, monomodal. Comparison with blend of 8-arm PEG (Shearwater, 10 kDa) and PEI (Aldrich, 800 Da): Mₙ=3100, M_{w}=15 000, Mₚ=12 000, PD=4.91, monomodal.

Investigations of the surface activity of the polymer of Example No. 27 were carried out by the method of Lecomte du Nouy (ring method) at 22° C. using a tensiometer. The surface tension of the solution in relation to air was measured. The instrument was calibrated with extra pure water, which was also employed as solvent for the polymer sample.
Measured data: σₘᵢₙ=56 mN/m, CMC=12 mg/ml.

The following can be prepared in the same way:

| | Starting compounds/homopolymers | | Structure |
|---|---|---|---|
| | Hydrophilic, | | of the block |
| No. | noninic polymer | Polyethyleneimine | copolymer |
| 28 | mPEG Mₙ ca. 5000 | IPEI Mₙ ca. 423 (Aldrich) | AB |
| 29 | (Aldrich) | bPEI Mₙ ca. 800 (Aldrich) | AB |
| 30 | | bPEI Mₙ ca. 2000 (Aldrich) | AB |
| 31 | IPEG Mₙ ca. 5000 | IPEI Mₙ ca. 423 (Aldrich) | ABA |
| 32 | (Aldrich) | bPEI Mₙ ca. 800 (Aldrich) | ABA |
| 33 | | bPEI Mₙ ca. 2000 (Aldrich) | ABA |
| 34 | 4-arm PEG MW | IPEI Mₙ ca. 423 (Aldrich) | AB₄ |
| 35 | ca. 15 000 | bPEI Mₙ ca. 800 (Aldrich) | AB₄ |
| 36 | (Shearwater) | bPEI Mₙ ca. 2000 (Aldrich) | AB₄ |
| 37 | 8-arm PEG MW | IPEI Mₙ ca. 423 (Aldrich) | AB₈ |
| 38 | ca. 10 000 | bPEI Mₙ ca. 800 (Aldrich) | AB₈ |
| 39 | (Shearwater) | bPEI Mₙ ca. 2000 (Aldrich) | AB₈ |
| 40 | Star PEG 429 MW | IPEI Mₙ ca. 423 (Aldrich) | AB₁₃ |
| 41 | ca. 250 000 | bPEI Mₙ ca. 800 (Aldrich) | AB₁₃ |
| 42 | (Shearwater) | bPEI Mₙ ca. 2000 (Aldrich) | AB₁₃ |
| 43 | α-Cyclodextrin | IPEI Mₙ ca. 423 (Aldrich) | AB₁₈ |
| 44 | (Aldrich) | bPEI Mₙ ca. 800 (Aldrich) | AB₁₈ |
| 45 | | bPEI Mₙ ca. 2000 (Aldrich) | AB₁₈ |
| 46 | β-Cyclodextrin | IPEI Mₙ ca. 423 (Aldrich) | AB₂₁ |
| 47 | (Aldrich) | bPEI Mₙ ca. 800 (Aldrich) | AB₂₁ |
| 48 | | bPEI Mₙ ca. 2000 (Aldrich) | AB₂₁ |
| 49 | γ-Cyclodextrin | IPEI Mₙ ca. 423 (Aldrich) | AB₂₄ |
| 50 | (Aldrich) | bPEI Mₙ ca. 800 (Aldrich) | AB₂₄ |
| 51 | | bPEI Mₙ ca. 2000 (Aldrich) | AB₂₄ |
| 52 | PVA 80% hydrolyzed | IPEI Mₙ ca. 423 (Aldrich) | ABₙ |
| 53 | M_{w} = 9000-10 000 | bPEI Mₙ ca. 800 (Aldrich) | ABₙ |
| 54 | | bPEI Mₙ ca. 2000 (Aldrich) | ABₙ |

### Example 55

### Preparation of a PEG-PEI Copolymer (Macroregulator Route)

1 g (0.2 mmol) of a monomethylated PEG (MW 5000 g/mol) which has an amino group at the other end of the chain is weighed into a 50 ml round-bottomed flask with magnetic stirring bar and reflux condenser, and is dissolved in 20 ml of distilled water. 2 ml (39 mmol) of ethyleneimine are added to this polymer solution. The polymerization is started with 200 μl (2 mmol) of dimethyl sulfate as initiator, and the mixture is heated at 60° C. for 8 days. The solvent is then removed under reduced pressure in order to redissolve the remaining mass in 20 ml of ethanol. The solution is added dropwise to 250 ml of diethyl ether, whereupon the polymer separates out. The polymer is isolated by filtration, and solvent residues are removed in the vacuum oven at 50° C. for 3 weeks. 1.9 g of a pale yellowish, resinous polymer are obtained (yield: 73%).

The following can be prepared in a similar way: (all amino-modified PEGs are obtainable from RAPP Polymere, Tübingen)

| | Starting compounds | | Molar | Structure |
|---|---|---|---|---|
| | Polyethylene | | ratio | of the block |
| No. | glycol | Polyethyleneimine | EG:EI | copolymer |
| 56 | CH₃O-PEG-NH₂ | Ethyleneimine | 1:1 | AB diblock |
| 57 | Mₙ ca. 2000 | | 1:2 | AB diblock |
| 58 | | | 1:10 | AB diblock |
| 59 | CH₃O-PEG-NH₂ | Ethyleneimine | 1:1 | AB diblock |
| 60p | Mₙ ca. 5000 | | 1:10 | AB diblock |
| 61 | CH₃O-PEG-NH₂ | Ethyleneimine | 1:1 | AB diblock |
| 62 | Mₙ 10 000 | | 1:2 | AB diblock |
| 63 | | | 1:10 | AB diblock |
| 64 | CH₃O-PEG-NH₂ | Ethyleneimine | 1:1 | AB diblock |
| 65 | Mₙ 20 000 | | 1:2 | AB diblock |
| 66 | | | 1:10 | AB diblock |

The polymers were characterized by ¹H and ¹³C NMR spectroscopy and gel permeation chromatography. The following data were obtained for Example No.56. They are representative of the other examples, for which very similar data were obtained.

¹H NMR (500 MHz, D₂O): δ/ppm=2.60-3.00 (ethyleneimine CH₂), 3.78 (ethylene glycol CH₂).

¹³C NMR (125 MHz, D₂O): δ/ppm=38.2 (ethyleneimine CH₂), 39.9 (ethyleneimine CH₂), 46.2 (ethyleneimine CH₂), 47.9 (ethyleneimine CH₂), 51.7 (ethyleneimine CH₂), 53.4 (ethyleneimine CH₂), 54.8 (ethyleneimine CH₂), 70.2 (ethylene glycol CH₂).

GPC (aminoethyl methacrylate gel, 1% formic acid, 0.5 ml/min, 25° C., calibrated using pullulan standards): Mₙ=21 000, M_{w}=40 000, Mₚ=16 000, PD=1.9, monomodal.

Comparison with CH₃O—PEG—NH₂ (RAPP Polymere, 5000 Da): Mₙ=9100, M_{w}=14 000, Mₚ=16 000, PD=1.6, monomodal.

### Example 67

### Preparation of a PEG-PEI Copolymer (Macroinitiator Route)

Preparation of the Macroinitiator

2 g (0.4 mmol, 1 eq.) of a monomethyl ether polyethylene glycol (Aldrich, MW 5000) are weighed into a 50 ml round-bottomed flask with magnetic stirring bar and reflux condenser and are dissolved in 25 ml of distilled chloroform. 0.31 g of tosyl chloride (1.6 mmol, 4 eq.) are added to the stirred polymer solution. Finally, 0.22 ml of triethylamine (0.16g, 1.6 mmol, 4 eq.) are added to the mixture as catalyst. The mixture is heated under reflux for 18 h. To isolate and purify the polymer, the solution is poured into 500 ml of diethyl ether. The precipitated polymer is filtered off, washed with a large amount of diethyl ether and dried in vacuo. 1.90 g of a white, flaky substance are obtained (91 % yield).

### Preparation of the PEG-PEI Block Copolymer

0.5 g of the macroinitiator (0.096 mmol, 1 eq.) is weighed into a 25 ml round-bottomed flask with magnetic stirring bar and reflux condenser and is dissolved in 10 ml of distilled water. While stirring, 1 ml of ethyleneimine (0.832 g, 19.32 mmol, 200 eq.) is added dropwise, and the mixture is heated at 60° C. for 24 h. The volatile components are removed under reduced pressure. A white, resinous substance remains and is redissolved in 10 ml of water and precipitated with 200 ml of tetrahydrofuran. The polymer is isolated by decantation and dried in vacuo. 0.95 g of a yellowish resinous substance is obtained (71% yield).

The following can be prepared in a similar way: (all monomethyl-PEGs are obtainable from Aldrich)

| | Starting compounds | | Molar | Structure |
|---|---|---|---|---|
| | Polyethylene | | ratio | of the block |
| No. | glycol | Polyethyleneimine | PEG:EI | copolymer |
| 68 | CH₃O-PEG-Ts | Ethyleneimine | 1:10 | AB diblock |
| 69 | Mₙ ca. 550 | | 1:50 | AB diblock |
| 70 | | | 1:200 | AB diblock |
| 71 | CH₃O-PEG-Ts | Ethyleneimine | 1:10 | AB diblock |
| 72 | Mₙ ca. 750 | | 1:50 | AB diblock |
| 73 | | | 1:200 | AB diblock |
| 74 | CH₃O-PEG-Ts | Ethyleneimine | 1:10 | AB diblock |
| 75 | Mₙ ca. 2000 | | 1:50 | AB diblock |
| 76 | | | 1:200 | AB diblock |
| 77 | CH₃O-PEG-Ts | Ethyleneimine | 1:10 | AB diblock |
| 78 | Mₙ ca. 5000 | | 1:50 | AB diblock |

The polymers were characterized by ¹H and ¹³C NMR spectroscopy and gel permeation chromatography. The following data were obtained for Example No. 67. They are representative of the other examples, for which very similar data were obtained.

¹H NMR (500 MHz, D₂O): δ/ppm=2.80-3.20 (ethyleneimine CH₂), 3.80 (ethylene glycol CH₂).

¹³C NMR (125 MHz, D₂O): δ/ppm=37.9 (ethyleneimine CH₂), 39.4 (ethyleneimine CH₂), 46.1 (ethyleneimine CH₂), 47.2 (ethyleneimine CH₂), 51.3-52.7 (ethyleneimine CH₂), 70.2 (ethylene glycol CH₂).

GPC (aminoethyl methacrylate gel), 1% formic acid, 0.5 ml/min, 25° C., calibrated using pullulan standards):
Mₙ=35 000, M_{w}=90 000, Mₚ=52 000, PD=2.6, monomodal.
Comparison with CH₃O—PEG—Ts 5000 Da): Mₙ=4800, M_{w}=7600, Mₚ=8600, PD=1.6, monomodal.

Abbreviations
bPEG branched polyethylene glycol
bPEI branched polyethyleneimine
CMC critical micelle concentration
DMF dimethylformamide
HMDI hexamethylene diisocyanate
IPEG linear polyethylene glycol
IPEI linear polyethyleneimine
Mₙ number average molecular weight
Mₚ peak molecular weight
mPEG monomethoxy polyethylene glycol
M_{w} weight average molecular weight
MW unspecified average molecular weight
PD polydispersity
Ts tosyl
σₘᵢₙ minimum surface tension

### Biological Examples

I. Transfection Experiments

The transfection properties of the polymers PEI(PEG)₂₀ (Example 1) and PEG(PEI)₈ (Example 27) were studied on the 3T3 cell line. 50 000 cells/well were seeded in 12 well plates and incubated for 24 hours (DMEM+2 mM glutamine+10% FCS, 37° C., 10% CO₂). The medium was then changed. 4 μg of pGL3 plasmid in 100 μl of 150 mM saline in each well were complexed with the appropriate amount of polymer in 100 μl of 150 mM saline and, after 10 minutes, added to the cells. After4 hours, the medium was again changed and, after 48 hours, the evaluation took place. Luciferase expression was determined using the Promega luciferase assay kit in a Berthold Sirius luminometer. The protein concentration was quantified with a modified BCA assay. The stated data are in each case the mean of three wells±standard deviation for the corresponding nitrogen/phosphorus ratios.

### Example 1

### [PEI(PEG)₂₀]

Measured data

N/P 5: 0.0057±0.0036 ng/mg of protein

N/P 10: 0.1786±0.1522 ng/mg of protein

N/P 20: 0.6952±0.5498 ng/mg of protein

N/P 50: 5.1963±2.6863 ng/mg of protein

(only plasmid: 0.0000±0.00004 ng/mg of protein)

### Example 27

### [PEG(PEI)₈]

Measured Data

N/P 5: 0.0024±0.0012 ng/mg of protein

N/P 10: 0.0045±0.0046 ng/mg of protein

N/P 20: 0.0109±0.0078 ng/mg of protein

N/P 50: 0.0765±0.0498 ng/mg of protein

(only plasmid: 0.0000±0.00004 ng/mg of protein)

In both cases it was possible to detect gene expression on the basis of transfection having taken place. Moreover, PEI(PEG)₂₀ shows a distinctly greater transfection efficiency than does PEG(PEI)₈.

II. In Vitro Cytotoxicity Determination by the MTT Assay

The copolymers of Examples 1 and 27 were studied for their cytotoxicity in the cell culture model using the MTT assay by the method of Mosmann (J. Immunol. Methods. 65:55-63 (1983)).8000 L929 mouse fibroblasts/well were preincubated in 96 wells for 24 h and treated with the polymer solutions at various concentrations for 3, 12 and 24 h. The mitochondrial activity was determined through the conversion of the MTT dye to the formazan, which was quantified by. spectrophotometry. The polymers were employed as solutions in DMEM with 10% FCS in five different concentrations. If necessary, the pH was adjusted to 7.4 and the samples were sterilized by filtration (0.2 μm). The blends were prepared by mixing the two individual components (subtracting the amount of spacer). For the evaluation, the cellular viability [%] was plotted against the polymer concentrations employed, and the IC50 was determined.

Result
The in vitro cytotoxicity of the free polymers increases with increasing polymer concentration and with increasing incubation time.
Copolymer of Example 1: The toxicity of the mixture of individual components PEI 25 kDa and PEG 550 Da corresponds to the toxicity of the free PEI 25 kDa. The tolerability is distinctly improved by the covalent linkage of the two components. Although the toxicity profile after 24 h corresponds to that of the individual components and thus to that of the free PEI 25 kDa, the cytotoxicity falls with shorter incubation periods. The PEG coating masks the positive charge of the polyethyleneimine, and thus the charge-mediated effects on cell membranes are reduced.
Copolymer of Example 27: The mixture of the two individual components PEI 700 Da and PEG 10 kDa showed no reduction in the viability of the cells up to 10 mg/ml. In the same concentration range, the copolymer showed an increased limitation on cellular viability after 3, 12 and 24 h, which can be explained by the increase in molecular weight.
Example 27 shows less cytotoxicity than Example 1.

III. In vitro Cytotoxicity Determination by the LDH Assay

L929 mouse fibroblasts were seeded in the same cell density as in the MTT assay in 6-well multidishes, preincubated for 48 h and incubated with the polymer solution (in PBS pH 7.4) for 1, 2, 3 and 6 h. The extracellular LDH fraction was quantified with a standard kit (Sigma, DG-1340-K) by photometric determination of the reduction of NAD in the presence of lactate and LDH. To determine the 100% value, cells were lyzed with 0.1 % Triton X-100.

Result

The LDH assay confirms the results of the MTT test. Correlation of the two assays shows that membrane damage starts first and, after a time lag, the reduction in metabolic activity starts. The membrane-damaging effect of the polymers becomes stronger as the incubation time and polymer concentration increase.

IV. DNA Binding of the Copolymers Determined by Agarose Gel Electrophoresis

The binding capacity of the copolymers of Examples 1 and 27 was determined by electrophoresis on 1% agarose gels at 80 V. The plasmids (CMV-nlacZ) are located by UV excitation at 254 nm after ethidium bromide staining.

Result
Both polymers are capable of electrostatic interaction with the plasmid.
Consistent with the blend, the polymer of Example 1 is able to bind plasmid completely from a nitrogen-PEI/phosphate-DNA ratio (N/P ratio) of 1.7 onwards. The ethidium bromide exclusion observed with the blend (from N/P 5.8), a sign of intensive DNA condensation, is incomplete for the copolymer up to N/P 23.0.
Whereas for the blend of Example 27 complete plasmid binding is to be observed only from N/P 4.1 onwards, and no complete ethidium exclusion is to be observed, the copolymer showed plasmid binding from N/P 2.4 onwards and exclusion of the dye from N/P 16.6 onwards.

V. Erythrocyte Aggregation Assay

Erythrocytes were isolated from the citrated blood of Wistar rats by the method of Parnham and Wetzig (Chem. Phys. Lipids, 1993, 64: 263-274), seeded in 24 wells and incubated with the test solutions at 37° C. for 2 h. The aggregation and adhesion of the erythrocytes under the influence of the polymerwere examined under the microscope. Untreated erythrocytes served as control.

Result
Free copolymer of Example 1 showed at concentrations of 0.27-18 μg/well by comparison with the blend and with PEI 25 kDa a reduced aggregation and adhesion of the red blood corpuscles to the cell culture dishes. Whereas no significant differences were to be seen at low concentrations (0.27-0.7 pg/well), a marked difference between copolymer and blend or PEI 25 kDa was detectable with increasing concentration. The aggregating effect increases as the N/P ratio increases.
Copolymer of Example 27 showed the opposite behavior. Aggregation of the blend and of free PEI is less pronounced than that of the copolymer.
The erythrocyte aggregation is significantly reduced through complexation of both copolymers with plasmid DNA compared with the free polymer.

VI. Hemolysis assay

Erythrocytes were isolated from the citrated blood of Wistar rats by the method of Parnham and Wetzig (Chem. Phys. Lipids, 1993, 64: 263-274), mixed with the polymer solutions and incubated at 37° C. for 1 h. The erythrocytes are pelleted by centrifugation (10 min, 25° C., 700 g), and the hemolyzate is measured by photometry on the supernatant at 540 nm.

Result
The individual components PEG 8-arm, PEG 500 Da and PEI 700 Da show no significant hemolytic effects in the concentration range 0.001-10 mg/ml (all 1-3%).
The copolymer of Example 27 likewise shows no pronounced effects (<5%) in the same concentration range.
With the individual components PEI 25 kDa and with the blend for Example 1, the hemolytic activity increases at 0.001-10 mg/ml (22.13% at 10 mg/ml).
The copolymer of Example 1 shows an increasing lytic activity of up to 13.30% up to 0.5 mg/ml, while the hemolytic effect decreases again at higher concentrations up to 10 mg/ml (2.90% at 10 mg/ml).

VII. Pharmacokinetics and Organ Distribution of Polymer-DNA Complexes in Mice

The pharmacokinetics and organ distribution of the copolymers of Example 1 and 27 were determined in balb/c mice. The polymers were radiolabeled with ¹²⁵I Bolton Hunter reagent (Pharmacia Biotech). Amounts of 0.4 or 0.04 or 0.008 mg of PEI (component) per kg of mouse were complexed with the appropriate amount of NF-KB decoy oligodeoxynucleotide (ODN) in the nitrogen/phosphorus ratio N/P 3.5 or N/P 6 in a total volume of 80 μl in 5% glucose solution and, after 10 minutes, injected into the anesthetized mice via the subclavian vein. After 20 seconds, 1, 2, 5, 15, 30, 60, 90 and 120 minutes, blood samples were taken from the arteria aorta communis through a catheter. The urine was collected through a bladder catheter for 120 minutes. After 120 minutes, the mice were decapitated and the organs cortex, kidney, liver, heart, lung, spleen and adipose tissue were removed. The amount of polymer in the samples was determined by measuring the radioactivity with a 1277 Gammamaster automatic gamma counter (LKB Wallac).

The data were analyzed using the Kinetica 1.1 program and a 2-compartment model for i.v. bolus injection. The volume of distribution (V_{c}), the elimination constant (kₑₗ) and AUC were calculated from the blood level plots. Mean±standard deviation are stated when three animals could be analyzed, the median is stated for two animals, and the value is stated in parentheses when there was only one animal.

Complex Preparation and Dosages

| | | Dose | V_{c} | kₑₗ | AUC |
|---|---|---|---|---|---|
| Polymer | N/P | [mg/kg] | [ml] | [min⁻¹] | [min μg ml⁻¹] |
| 25 kDa PEI | 3.5:1 | 0.4 | 23.39 | 0.106 | 4.89 |
| Example 1 | 3.5:1 | 0.4 | (4.54) | (0.028) | (79.03) |
| Example 27 | 3.5:1 | 0.4 | 5.84 ± 0.4 | 0.104 ± 0.017 | 16.86 ± 1.64 |
| 25 kDa PEI | 6:1 | 0.4 | 5.39 | 0.099 | 19.22 |
| 25 kDa PEI | 6:1 | 0.04 | 1.37 ± 0.2 | 0.14 ± 0.026 | 6.22 ± 1.18 |
| 25 kDa PEI | 6:1 | 0.008 | 9.57 ± 1.78 | 0.063 ± 0.009 | 0.34 ± 0.1 |
| Example 1 | 6:1 | 0.4 | 6.20 | 0.067 | 27.84 |
| Example 1 | 6:1 | 0.04 | 3.37 ± 0.32 | 0.072 ± 0.01 | 4.0 ± 0.67 |
| Example 1 | 6:1 | 0.008 | 5.1 ± 0.55 | 0.054 ± 0.004 | 0.80 ± 0.10 |
| Example 27 | 6:1 | 0.4 | 8.12 | 0.0593 | 21.72 |

Result
Observations with a relatively low dose indicate that the toxicity of PEI(PEG)₂₀ is weaker than that of PEI 25 kDa.
The plasma levels of all the polymers could be described by a 2-compartment model.
The copolymers have a higher AUC and a smaller volume of distribution than the 25 kDA PEI. PEI(PEG)₂₀ (Example 1) has a larger effect than PEG(PEI)₈ (Example 27).
Elimination was reduced with the copolymers.
V_{c} and kₑₗ show no detectable dose-dependency.
The calculated AUC for PEI 25 kDa and Example 1 was proportional to the dose, while the gradient of the AUC/dose lines was larger with the copolymer of Example 1.
The main organs of distribution after 120 minutes were liver, kidney and spleen. For the 6:1 complexes, the copolymers show a reduced uptake in liver and spleen and a higher uptake in the kidney compared with PEI 25 kDa.

That which is claimed:

1. A compound of the formula II
C(—Y—D)ₘ (II)
in which
C is a linear or branched PEI with a molecular weight of from 100 to 1 000 000 g/mol further comprising a moiety [A⁻]_{w}, where A⁻ is an equivalent of an anion and w is an integer selected to balance the positive charges in the polyethyleneimine (PEI);
D is a residue of a polyethylene glycol of the formula
—(CH₂CH₂O)_{n′}—R¹
which is bonded via 0 and in which n′ is from 3 to 25 000, and R¹ is hydrogen, an aliphatic radical or another OH-protective group or a cellular ligand;
Y is a direct linkage of blocks C and D or a linker with the following structures whose C-terminal side is linked to a nitrogen atom of the PEI:
—C(O)NH(CH₂)ₒNHC(O)O— with o=1 to 20,
—C(O)NH(aryl)NHC(O)O— with aryl=aromatic unit,
—(CH₂)ₜC(O)O— with t=2 to 10,
—CH₂CH(OH)CH₂O—, or
—(CH₂)O— with u=1 to 20;
m is an integer from 1 to 200;
and the molar ratio between C and D is between 1 to 20 and 1 to 100.

2. A compound as claimed in claim 1, in which
C is a linear or branched PEI with a molecular weight of from 400 to 100 000 g/mol further comprising a moiety [A⁻]_{w}, where A⁻ is an equivalent of an anion and w is an integer selected to balance the positive charges in the polyethyleneimine (PEI);
D is a residue of a polyethylene glycol of the formula
—(CH₂CH₂O)ₙ,—R¹
which is bonded via O and in which n′ is from 10 to 5000, and R¹ is hydrogen, an aliphatic radical or another OH-protective group or a cellular ligand;
Y is a direct linkage of blocks C and D or a linker with the following structures whose C-terminal side is linked to a nitrogen atom of the PEI:
—C(O)NH(CH₂)ₒNHC(O)O— with o=2 to 10,
—C(O)NH(aryl)NHC(O)O— with aryl=aromatic unit with one nucleus,
—(CH₂)ₜC(O)O— with t=2 to 3,
—CH₂CH(OH)CH₂O—, or
—(CH₂)ᵤO— with u=1 to 6;
m is an integer from 1 to 100
and the molar ratio between C and D is between 1 to 20 and 1 to 100.

3. A compound as claimed in claim 1, in which
C is a linear or branched PEI with a molecular weight of from 400 to 50 000 g/mol further comprising a moiety [A⁻]_{w}, where A⁻ is an equivalent of an anion and w is an integer selected to balance the positive charges in the polyethyleneimine (PEI);
D is a residue of a polyethylene glycol of the formula
—(CH₂CH₂O)_{n′}—R¹
which is bonded via O and in which n′ is from 10 to 1000, and R¹ is hydrogen, an aliphatic radical or another OH-protective group or a cellular ligand;
Y is a direct linkage of blocks C and D or a linker with the following structures whose C-terminal side is linked to a nitrogen atom of the PEI:
—C(O)NH(CH₂)ₒNHC(O)O— with o=4 to 6,
—C(O)NH(aryl)NHC(O)O— with aryl=tolyl,
—(CH₂)ₜC(O)O— with t=2,
—CH₂CH(OH)CH₂O—, or
—(CH₂)ᵤO— with u=1 to 3;
m is an integer from 1 to 50;
and the molar ratio between C and D is between 1 to 20 and 1 to 100.

4. The method of complexation of polynucleic acids in aqueous systems which comprises contacting a compound of the formula II in which Y is a linker of the formula —C(O)NH(CH₂)ₒNHC(O)O— with o=1-10, and the other radicals are as defined in claim 1 with a polynucleic acid.

5. A process for preparing compounds of the formula II as claimed in claim 1, which comprises initially reacting compounds of the general formula IX
D—OH, with D as defined in formula II, (IX)
with diisocyanate and subsequently reacting the resulting compound with linear or branched polyethyleneimine.

6. The method of complexation of polynucleic acids in aqueous systems which comprises contacting a compound of the formula II
C(—Y—D)ₘ (II)
in which
C is a linear or branched PEI with a molecular weight of from 100 to 1 000 000 g/mol further comprising a moiety [A⁻], where A⁻ is an equivalent of an anion and w is an integer selected to balance the positive charges in the polyethyleneimine (PEI);
D is a residue of a polyethylene glycol of the formula
—(CH₂CH₂O)_{n′}—R¹
which is bonded via O and in which n′ is from 3 to 25 000, and R¹ is hydrogen, an aliphatic radical or another OH-protective group or a cellular ligand;
Y is a direct linkage of blocks C and D or a linker with the following structures whose C-terminal is linked to a nitrogen atom of the PEI:
—C(O)NH(CH₂)ₒNHC(O)O— with o=1 to 20,
—C(O)NH(aryl)NHC(O)O— with aryl=aromatic unit,
—(CH₂)ₜC(O)O— with t=2 to 10,
—CH₂CH(OH)CH₂O—, or
—(CH₂)ᵤO— with u=1 to 20,
m is an integer from 1 to 200
and the molar ratio between C and D is between 1 to 20 and 1 to 100 with a polynucleic acid.

7. The method as claimed in claim 6, wherein a compound of the formula II is used, in which
C is a linear or branched PEI with a molecular weight of from 400 to 100 000 g/mol further comprising a moiety [A⁻]_{w}, where A⁻ is an equivalent of an anion and w is an integer selected to balance the positive charges in the polyethyleneimine (PEI);
D is a residue of a polyethylene glycol of the formula
—(CH₂CH₂O)_{n′—R}¹
which is bonded via O and in which n′ is from 10 to 5000, and R¹ is hydrogen, an aliphatic radical or another OH-protective group or a cellular ligand;
Y is a direct linkage of blocks C and D or a linker with the following structures whose C-terminal side is linked to a nitrogen atom of the PEI:
—C(O)NH(CH₂)ₒNHC(O)O— with o=2 to 10,
—C(O)NH(aryl)NHC(O)O— with aryl=aromatic unit with one nucleus,
—(CH₂)ₜC(O)O— with t=2 to 3,
—CH₂CH(OH)CH₂O—, or
—(CH₂)ᵤO— with u=1 to 6;
m is an integer from 1 to 100;
and the molar ratio between C and D is between 1 to 20 and 1 to 100.

8. The method as claimed in claim 6, wherein a compound of the formula II is used, in which
C is a linear or branched PEI with a molecular weight of from 400 to 50 000 g/mol further comprising a moiety [A⁻]_{w}, where A⁻ is an equivalent of an anion and w is an integer selected to balance the positive charges in the polyethyleneimine (PEI);
D is a residue of a polyethylene glycol of the formula
—(CH₂CH₂O)ₙ,—R¹
which is bonded via O and in which n′ is from 10 to 1000, and R¹ is hydrogen, an aliphatic radical or another OH-protective group or a cellular ligand;
Y is a direct linkage of blocks C and D or a linker with the following structures whose C-terminal side is linked to a nitrogen atom of the PEI:
—C(O)NH(CH₂)ₒNHC(O)O— with o=4 to 6,
—C(O)NH(aryl)NHC(O)O— with aryl=tolyl,
—(CH₂)ₜC(O)O— with t=2,
—CH₂CH(OH)CH₂O—, or
—(CH₂)ᵤO— with u=1 to 3;
m is an integer from 1 to 50
and the molar ratio between C and D is between 1 to 20 and 1 to 100.

9. The method of claim 6, wherein the polynucleic acid is DNA.

10. A composition which comprises at least one nucleic acid and one compound of the formula II according to claim 1.

11. A compound according to claim 1, wherein
C is a star-shaped PEI with a molecular weight of from 100 to 1 000 000 g/mol and
D is a residue of a polyethylene glycol of the formula —(CH₂CH₂O)_{n′}—R¹ which is bonded via O and in which n′ is from 3 to 25 000, and R¹ is methyl or a cellular ligand.

12. A compound according to claim 1, wherein
C is a star-shaped PEI with a molecular weight of from 100 to 1 000 000 g/mol and
D is a residue of polyethylene glycol of the formula —(CH₂CH₂O)_{n′}—R¹ which is bonded via O and in which n′ is from 3 to 25 000, and R¹ is methyl.

13. A complex comprising
(i) a compound of the formula II
C(—Y—D)ₘ (II)
in which
C is a linear or branched PEI with a molecular weight of from 100 to 1 000 000 g/mol further comprising a moiety [A⁻]_{w}, where A⁻ is an equivalent of an anion and w is an integer selected to balance the positive charges in the polyethyleneimine (PEI);
D is a residue of a polyethylene glycol of the formula
—(CH₂CH₂O)_{n′}—R¹
which is bonded via O and in which n′ is from 3 to 25 000, and R¹ is hydrogen, an aliphatic radical or another OH-protective group or a cellular ligand;
Y is —C(O)NH(CH₂)ₒNHC(O)O— with o=1 to 20;
m is an integer from 1 to 200;
the molar ratio between C and D is between 1 to 20 and 1 to 100 and
(ii) a polynucleic acid.
